(19) United States
(12) Patent Application Publication
(10) Pub. No.: US 2021/0379121 A1
(43) Pub. Date: Dec. 9, 2021

(54) PROBIOTICS AND PROBIOTIC COMPOSITIONS HAVING MODIFIED CARBOHYDRATE METABOLISM

(71) Applicant: UNIVERSITY OF PITTSBURGH - OF THE COMMONWEALTH SYSTEM OF HIGHER EDUCATION, Pittsburgh, PA (US)

(72) Inventors: Abhinav Prakash Acharya, Pittsburgh, PA (US); Steven R. Little, Allison Park, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 17/410,360

(22) Filed: Aug. 24, 2021

Related U.S. Application Data
(63) Continuation of application No. PCT/US2020/019907, filed on Feb. 26, 2020.
(60) Provisional application No. 62/810,869, filed on Feb. 26, 2019.

(51) Int. Cl. A61K 35/741

(52) U.S. Cl. CPC A61K 35/741

(57) ABSTRACT

The present disclosure relates to probiotics and probiotic compositions having modified carbohydrate metabolism (e.g., modifying or metabolizing carbohydrates, or reduced glucose production), and thus regulating the absorption of the carbohydrates by the host subject. The present disclosure also relates to methods of making the probiotics and probiotic compositions. The present disclosure further relates to methods of regulating body weight in a subject, and methods of treating carbohydrate metabolism disorder, using the probiotics and probiotic compositions disclosed herein.

### 1. CROSS-REFERENCE TO RELATED APPLICATIONS

This Application is a continuation of International Patent Application No. PCT/US2020/019907, filed on Feb. 26, 2020, which claims priority to U.S. Provisional Application No. 62/810,869, filed on Feb. 26, 2019, the contents of which are incorporated by reference in their entireties.

### 2. INTRODUCTION

The present disclosure relates to probiotics and probiotic compositions having modified carbohydrate metabolism (e.g., modifying or metabolizing carbohydrates or reduced glucose production), and thus regulating the absorption of the carbohydrates by the host subject. The present disclosure also relates to methods of making the probiotics and probiotic compositions. The present disclosure further relates to methods of regulating body weight in a subject, and methods of treating carbohydrate metabolism disorder, using the probiotics and probiotic compositions disclosed herein.

### 3. BACKGROUND

Metabolic disorders have reached epidemic proportions. Worldwide, 1.9 billion adults are overweight or obese, while 462 million are underweight. See World health statistics 2018. Geneva: World Health Organization. More than 50 million children under 5 years of age are underweight, while 41 million are overweight or obese Id. The unbalanced caloric intake has been associated and linked to different disorders. For example, undernutrition can lead to heart failure and respiratory failure and to impair immune response while obesity can lead to type 2 diabetes, cardiovascular diseases, hypertension, stroke and certain forms of cancer.

Carbohydrate metabolism disorders are a group of metabolic disorders, where the patients cannot produce active enzymes to uptake, synthesize or break down the carbohydrates. Carbohydrate metabolism disorders may cause a harmful amount of sugar to build up in patients' bodies. That can lead to health problems, some of which can be serious. Some of the disorders are fatal.

Therefore, there remains a need for interventions of effectively regulating carbohydrate metabolism and treating carbohydrate metabolism disorders.

### 4. SUMMARY

The present disclosure relates to probiotics and probiotic compositions having modified carbohydrate metabolism (e.g., modifying or metabolizing carbohydrates, or reduced glucose production), and thus regulating the absorption of the carbohydrates by the host subject. The present disclosure also relates to methods of making the probiotics and probiotic compositions. The present disclosure further relates to methods of regulating body weight in a subject, and methods of treating carbohydrate metabolism disorder, using the probiotics and probiotic compositions disclosed herein.

In one aspect, the present disclosure provides a probiotic composition comprising probiotic bacteria, wherein the probiotic bacteria have a modified carbohydrate metabolism. In certain embodiments, the probiotic bacteria produce a change in glucose production as compared to control probiotic bacteria. In certain embodiments, the probiotic bacteria increase carbohydrate absorption by a host subject. In certain embodiments, the probiotic bacteria reduce carbohydrate absorption by a host subject.

In certain embodiments, the probiotic bacteria survive in a culture media, wherein at least about 60%, at least about 70%, at least about 80%, at least about 90%, at least about 95%, at least about 99%, or at least about 100% of carbohydrates in the culture media are starches. In certain embodiments, at most about 40%, at most about 30%, at most about 20%, at most about 10%, at most about 5%, at most about 1%, or about 0% of carbohydrates in the culture media are sugars.

In certain embodiments, the probiotic composition is for use in regulating a body weight of a subject. In certain embodiments, the probiotic composition is for use in increasing or decreasing the body weight of the subject. In certain embodiments, the subject has an underweight body weight mass (BMI). In certain embodiments, the probiotic composition is for use in maintaining or reducing the body weight of the subject. In certain embodiments, the subject has a healthy BMI, an overweight BMI, or an obese BMI. In certain embodiments, the probiotic composition is for use in treating a subject having a disorder of carbohydrate metabolism. In certain embodiments, the probiotic composition is for use in treating a subject having a genetic disorder of carbohydrate metabolism. In certain embodiments, the probiotic composition is for use in treating a subject having an eating disorder.

In another aspect, the present disclosure provides a kit comprising the probiotic composition disclosed herein.

In one aspect, the present disclosure provides a method of making a probiotic composition comprising:
(a) obtaining a microbiota sample from a subject;
(b) subjecting the microbiota sample to a starch-stress directed evolution to generate probiotic bacteria, wherein the probiotic bacteria have a modified carbohydrate metabolism as compared to control probiotic bacteria, wherein the control probiotic bacteria are not subject to starch-stress directed evolution; and
(c) incorporating the probiotic bacteria to the probiotic composition.

In certain embodiments, the microbiota sample is a saliva sample or a stool sample.

In certain embodiments, the probiotic bacteria produce a change in glucose production as compared to the control probiotic bacteria. In certain embodiments, the probiotic bacteria increase carbohydrate absorption by a subject. In certain embodiments, the probiotic bacteria reduce carbohydrate absorption by a subject. In certain embodiments, the probiotic bacteria survive in a culture media, wherein at least about 60%, at least about 70%, at least about 80%, at least about 90%, at least about 95%, at least about 99%, or at least about 100% of carbohydrates in the culture media are starches. In certain embodiments, at most about 40%, at most about 30%, at most about 20%, at most about 10%, at most about 5%, at most about 1%, or about 0% of carbohydrates in the culture media are sugars. In certain embodiments, the starch-stress directed evolution comprises culturing the microbiota sample in increasing starch concentrations.

In one aspect, the present disclosure provides a method of regulating body weight of a subject, comprising:
(a) obtaining a microbiota sample;
(b) subjecting the microbiota sample to a starch-stress directed evolution to generate probiotic bacteria, wherein the probiotic bacteria have a modified carbohydrate metabolism as compared to control probiotic bacteria, wherein the control probiotic bacteria are not subject to starch-stress directed evolution; and
(c) administering to the subject an effective amount of the probiotic bacteria.

In one aspect, the present disclosure provides a method of regulating body weight of a subject, comprising:
(a) obtaining a microbiota sample;
(b) isolating bacteria from the microbiota sample;
(c) subjecting the isolated bacteria to a starch-stress directed evolution to generate probiotic bacteria, wherein the probiotic bacteria have a modified carbohydrate metabolism as compared to control probiotic bacteria, wherein the control probiotic bacteria are not subject to starch-stress directed evolution; and
(d) administering to the subject an effective amount of the probiotic bacteria.

In certain embodiments, the microbiota sample is a saliva sample or a stool sample. In certain embodiments, the microbiota sample is obtained from the subject. In certain embodiments, the probiotic bacteria produce a change in glucose production as compared to the control probiotic bacteria. In certain embodiments, the probiotic bacteria increase carbohydrate absorption by the subject. In certain embodiments, the probiotic bacteria reduce carbohydrate absorption by the subject.

In certain embodiments, the probiotic bacteria survive in a culture media, wherein at least about 60%, at least about 70%, at least about 80%, at least about 90%, at least about 95%, at least about 99%, or at least about 100% of carbohydrates in the culture media are starches. In certain embodiments, at most about 40%, at most about 30%, at most about 20%, at most about 10%, at most about 5%, at most about 1%, or about 0% of carbohydrates in the culture media are sugars. In certain embodiments, the starch-stress directed evolution comprises culturing the microbiota sample in increasing starch concentrations. In certain embodiments, the method regulates a body weight of a subject. In certain embodiments, the method increases or reduces the body weight of the subject. In certain embodiments, the subject has an underweight BMI. In certain embodiments, the method maintains or reduces the body weight of the subject. In certain embodiments, the subject has a healthy BMI, an overweight BMI, or an obese BMI. In certain embodiments, the method is for use in treating a subject having a disorder of carbohydrate metabolism. In certain embodiments, the method is for use in treating a subject having a genetic disorder of carbohydrate metabolism. In certain embodiments, the method is for use in treating a subject having an eating disorder.

### 5. BRIEF DESCRIPTION OF THE DRAWINGS

FIG. 1 provides a diagram showing that evolved bacteria under starch stress consume starch at the same degree as non-evolved bacteria but the glucose production of the evolved bacteria is at a much smaller degree than the glucose production from non-evolved bacteria.

FIG. 2 provides a diagram showing that Streptococcus mitis, Streptococcus pneumoniae, and Streptococcus pseudopneumoniae were found as the most common stains.

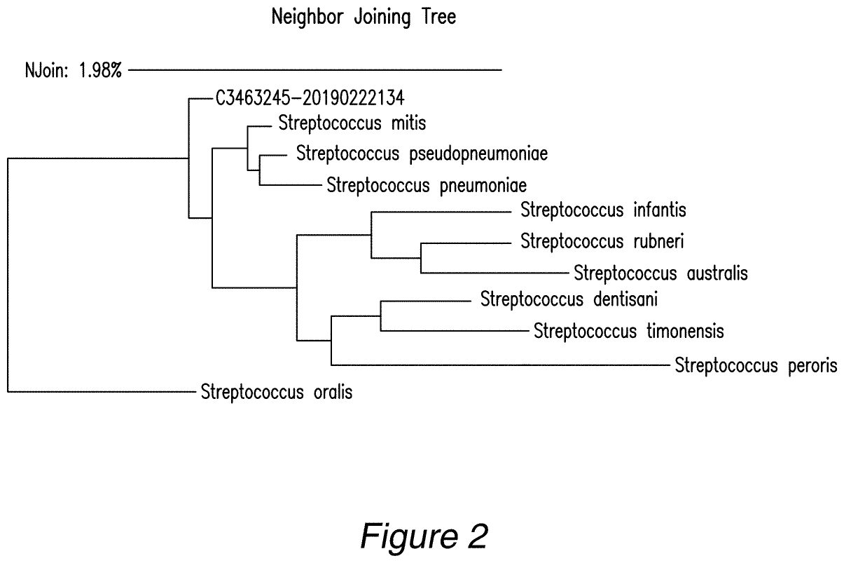

FIG. 3 shows representative images of starch digesting colonies that generated halo on an agar plate after Lugol test. Colonies are indicated in the boxes.

FIG. 4 provides a graph showing the survival of bacteria during (67% starch) and after (100% starch) evolution process.

FIG. 5 provides a graph showing that evolved bacteria had modified carbohydrate metabolism as indicated by changes in glucose production.

### 6. DETAILED DESCRIPTION

The present disclosure relates to probiotics and probiotic compositions having modified carbohydrate metabolisms, e.g., modifying, metabolizing, or storing carbohydrates before the carbohydrates are absorbed by a host subject, changed glucose production after carbohydrates are consumed by the probiotics. Thus, the probiotics and probiotic compositions disclosed herein can regulate the absorption of the carbohydrate by the host subject, e.g. at the gastrointestinal tract, intestines, or small intestine. The present disclosure further relates to methods of using the probiotics and probiotic compositions disclosed herein to regulate (e.g., increasing, maintaining or reducing) body weight in a subject; treat a subject having underweight or undernutrition, overweight or obesity, eating disorders, or disorders of carbohydrate metabolism. Non-limiting embodiments of the invention are described by the present specification and Examples.

For purposes of clarity of disclosure and not by way of limitation, the detailed description is divided into the following subsections:
5.1 Definitions;
5.2 Probiotics and Probiotic Compositions;
5.3 Methods of Making;
5.4 Methods of Use; and
5.5 Kits

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.”

The terms “having,” “including,” “containing” and “comprising” are interchangeable and one of skill in the art is cognizant that these terms are open ended terms. As used herein, these terms are intended to cover a non-exclusive inclusion, such that a process, method, article, or apparatus that comprises a list of elements does not include only those elements but can include other elements not expressly listed or inherent to such process, method, article, or apparatus.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” of a substance as that term is used herein is that amount sufficient to effect beneficial or desired results, including clinical results, and, as such, an “effective amount” depends upon the context in which it is being applied. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

As used herein, the term “bacteria” encompasses both prokaryotic organisms and archaea present in mammalian microbiota. The terms “intestinal microbiota”, “gut flora”, and “gastrointestinal microbiota” are used interchangeably to refer to bacteria in the digestive tract. The terms “saliva microbiota,” “saliva flora,” “mouth microbiota,” and “mouth flora” are used interchangeably to refer to bacteria found in the oral cavity. As used herein, the term “abundance” refers to the representation of a given phylum, order, family, or genera of microbe present in the digestive tract of a subject.

The terms “modified carbohydrate metabolism” or “change in carbohydrate metabolism” are used interchangeably to refer to a change in the carbohydrate metabolism of a probiotic bacterium. The change can be a decrease or an increase in the metabolism or in one or more metabolic pathways of carbohydrate of the isolated microbiota of a microbial species, genus, family, strain, order, or class. In certain embodiments, a modified carbohydrate metabolism or a change in carbohydrate metabolism result in a change (e.g., an increase or a decrease) in glucose production by a probiotic bacterium.

As used herein, the term “probiotics” or “probiotic bacteria” refers living bacteria that can be administered to a subject, e.g., orally consumed by a subject. In certain embodiments, the presently disclosed probiotic bacteria or probiotics reduce or increase the absorption of dietary carbohydrates at the gastrointestinal tract of a host subject. In certain embodiments, the presently disclosed probiotic bacteria or probiotics have the beneficial effects of modulating (e.g., maintaining, increasing or reducing) the body weight of a subject. In certain embodiments, the presently disclosed probiotic bacteria or probiotics have the beneficial effects of treating an overweight or obese subject. In certain embodiments, the presently disclosed probiotic bacteria or probiotics have the beneficial effects of treating an underweight or undernutrition subject. In certain embodiments, the presently disclosed probiotic bacteria or probiotics have the beneficial effects of treating a subject having eating disorders or disorders of carbohydrate metabolism.

In certain embodiments, probiotics having modified carbohydrate metabolism can decrease quantities of energy molecules in the gastrointestinal (GI) tract when administered to a subject, thus decreasing carbohydrate absorption by the subject. In certain embodiments, probiotics having modified carbohydrate metabolism can modify the dietary carbohydrates, where the modified carbohydrates cannot be or are difficult to be absorbed and/or metabolized by the subject. In certain embodiments, the probiotics having modified carbohydrate metabolism can consume carbohydrates in the GI tract in a way that increases the production of glucose as compared to other bacteria in the GI tract, which leads to increased glucose, and thus energy, absorption by the host subject. Therefore, change of the carbohydrate metabolism of the probiotics can lead to modification of the content of GI tract and modulation of the amount of carbohydrates that can be absorbed by the subject.

The term “stress-based directed evolution” of probiotics can refer to an ex-vivo introduction of environmental stressors to the isolated bacteria to enrich the bacteria for the desired trait and encourage the bacteria to enhance their protein production that favors their survival in the presence of stressors. The bacteria are then tested for the desired trait. The steps of screening and stressing can be repeated until the isolated bacteria can survive and proliferate in the presence of stressors. Non-limiting Exemplary stressors are carbohydrates, e.g., starches, or gelatinized starches.

As used herein, a “culture” of bacteria can refer to an in vitro culture of at least one bacterium species. Such bacteria can be cultured with one or more activators or repressors. As used herein, the terms “activators” and “repressors” refer to agents that increase or decrease the number and/or activity and/or metabolism of one or more desired bacteria, respectively.

As used herein, the term “probiotic composition” can refer to a composition containing at least one species, genus, family, strain, order, or class of probiotic bacteria (e.g., a single isolate or a combination of desired bacteria), and can also include any additional carriers, excipients, and/or therapeutic agents that can be administered to a mammal. In certain embodiments, the probiotic composition comprises a buffering agent to allow the probiotic bacteria to survive in the acidic environment of the stomach, that is, the probiotic bacteria resist low pH and are able to survive passage through the stomach to colonize and grow in the intestinal milieu. Buffering agents can include, for example, sodium bicarbonate, milk, yogurt, infant formula, and other dairy products. In certain embodiments, the probiotic composition is formulated as a food additive. In certain embodiments, the probiotic composition includes other materials known in the art for inclusion in food additives, such as water or other aqueous solutions, starch, binders, thickeners, colorants, flavorants, odorants, acidulants (e.g., lactic acid or malic acid, among others), vitamins, or minerals, among others.

The term “carrier” can refer to a diluent, adjuvant, excipient, or vehicle with which probiotic bacteria can be administered. Such carriers can be, for example, sterile liquids, such as water and oils, including those of petroleum, animal, vegetable or synthetic origin, such as peanut oil, soybean oil, mineral oil, sesame oil and the like. Water or aqueous solution, saline solutions and aqueous dextrose and glycerol solutions can be employed as carriers, particularly for injectable solutions. In certain embodiments, the carrier can be a solid dosage form carrier, including but not limited to one or more of a binder (for compressed pills), a glidant, an encapsulating agent, a flavorant, and a colorant. Suitable carriers are described, for example, in “Remington's Pharmaceutical Sciences” by E. W. Martin.

The terms “inhibiting,” “reducing” or “prevention,” or any variation of these terms, referred to herein, includes any measurable decrease or complete inhibition to achieve a desired result. The benefit to a subject to be treated is either statistically significant or at least perceptible to the patient or to the physician. Treatment includes partial or full resolution of symptoms associated with the medical condition to be treated.

The terms “increasing” or “inducing,” or any variation of these terms, referred to herein, includes any measurable increase or complete activation to achieve a desired result. The benefit to a subject to be treated is either statistically significant or at least perceptible to the patient or to the physician. Treatment includes partial or full resolution of symptoms associated with the medical condition to be treated.

In certain embodiments, a medical condition that can be treated by the presently disclosed methods and compositions is a weight-related condition, including, but not limited to, undernutrition or underweight (Body Mass Index (BMI) of about 18.5 or lower), obesity (BMI of about 30 or higher), overweight (BMI of about 25 to about 30), and associated medical conditions, and/or conditions where imbalanced caloric intake can be a risk factor of certain diseases including, but not limited to, hyperlipidemia, cancer, type 2 diabetes, hypertension, stroke, osteoarthritis, coronary heart disease, sleep apnea and respiratory problems, depression, and gallbladder disease.

In certain embodiments, an obese subject has a BMI of about 30 or higher, an overweight subject has a BMI of about 25 to about 30, a healthy and/or normal subject has a BMI of about 18.5 to about 25, and an underweight subject has a BMI of about 18.5 or lower. A BMI of about 18.5 to about 25 is considered healthy. In certain embodiments, a treatment of a subject (e.g., and/or a healthy subject or a subject having a medical condition) comprises maintaining the body weight of the subject and/or the healthy subject and/or maintaining a BMI of about 18.5 to about 25 of the subject In certain embodiments, a treatment of a subject comprises increasing the body weight of the subject and/or increasing a the BMI of about 18.5 or lower for the subject who has BMI of about 18.5 or lower. In certain embodiments, a treatment of a subject comprises reducing the bodyweight of the subject and/or reducing the BMI of the subject who has BMI of about 30 or higher, or about 25 to about 30.

In certain embodiments, a medical condition that can be treated by the presently disclosed methods and compositions is a genetic disorders of carbohydrate metabolism, e.g., including, but not limited to, disorders of gluconeogenesis, pyruvate carboxylase deficiency, phosphoenolpyruvate carboxykinase deficiency, or and glucose-6-phosphatase deficiency. In certain embodiments, a medical condition that can be treated by the presently disclosed methods and compositions is an eating disorder, including, but not limited to, e.g., anorexia nervosa, bulimia nervosa, avoidant/restrictive food intake disorder, and rumination disorder.

In certain embodiments, a medical condition that can be treated by the presently disclosed methods and compositions is hypoglycemia. In certain embodiments, the hypoglycemia can be caused by a pancreatic islet cell tumor, e.g., an insulinoma. In certain embodiments, a subject has recurrent hypoglycemic events. In certain embodiments, treatment of the subject comprises reducing the number of hypoglycemic events of the subject.

A “microbiome” as used herein can refer to the totality of microbes and their genetic elements (genomes) from a defined environment. A defined environment can, for example, be the intestine and/or the oral cavity of a human being. Thus, microbiome can include all area-specific microbiota and their complete genetic elements.

### 5.2 Probiotics and Probiotic Compositions

The presently disclosed subject matter relates to probiotics having a modified carbohydrate metabolism. In certain embodiments, the probiotics actively modulate processing of carbohydrate molecules and, therefore, their absorption at intestines.

In certain embodiments, the probiotics comprise personalized probiotics having modified carbohydrate metabolism, where the probiotics consume carbohydrates in the gastrointestinal tract of a subject and thus decrease the amount of carbohydrates that can be absorbed in the gastrointestinal tract of a subject. In certain embodiments, the probiotics metabolize or modify carbohydrates in a way that the modified carbohydrates cannot be absorbed in the gastrointestinal tract of a subject. In certain embodiments, the probiotics metabolize carbohydrates in a way to produce less glucose than control bacteria.

In certain embodiments, the probiotics metabolize carbohydrates in a way to produce more glucose than control bacteria.

In certain embodiments, the control bacteria do not have a modified carbohydrate metabolism. In certain embodiments, the control bacteria have not been subject to stress directed evolution.

The present disclosure further provides probiotic compositions comprising probiotic bacteria having modified carbohydrate metabolism as disclosed herein.

In certain embodiments, the probiotic composition is formulated as a food additive. In certain embodiments, the food additive disclosed herein further comprises other materials known in the art for inclusion in food additives, including, but not limited, water or other aqueous solutions, starch, binders, thickeners, colorants, flavorants, odorants, acidulants (e.g., lactic acid or malic acid, among others), vitamins, minerals, and combinations thereof. In certain embodiments, the food additive comprises between about 10³ and about 10⁴ CFU probiotic bacteria per gram of the food additive, between about 10⁴ and about 10⁵ CFU probiotic bacteria per gram of the food additive, between about 10⁵ and about 10⁶ CFU probiotic bacteria per gram of the food additive, between about 10⁶ and about 10⁷ CFU probiotic bacteria per gram of the food additive.

The present disclosure also provides a fortified food comprising the probiotics or probiotic compositions disclosed herein. In certain embodiments, the fortified food disclosed herein further comprises a base food. In certain embodiments, the food additive can be incorporated to a base food to form the fortified food. Any base foods known in the art can be used with the present disclosure. Non-limiting examples of base foods include kefir, yakult, miso, natto, tempeh, kimchee, sauerkraut, water, milk, fruit juices, vegetable juices, yogurt, carbonated soft drinks, non-carbonated soft drinks, coffee, tea, beer, wine, liquor, alcoholic mixed drinks, bread, cakes, cookies, crackers, extruded snacks, soups, frozen desserts, fried foods, pasta products, potato products, rice products, corn products, wheat products, dairy products, confectionaries, hard candies, nutritional bars, breakfast cereals, bread dough, bread dough mix, sauces, processed meats, and cheeses.

Administration of the probiotic composition disclosed herein can be accomplished by any method likely to introduce the bacteria into the desired location. In certain embodiments, the probiotics can be administered to a subject, in the form of a food additive or a fortified food disclosed herein, by oral consumption. In certain embodiments, the probiotic bacteria can be mixed with a carrier and (for easier delivery to the digestive tract) be applied to liquid or solid food, feed, or drinking water. The carrier material should be non-toxic to the bacteria and the subject/patient. In certain embodiments, the carrier contains an ingredient that promotes viability of the bacteria during storage. The formulation can include added ingredients to improve palatability and improve shelf-life. If a reproducible and measured dose is desired, the bacteria can be administered by a rumen cannula.

In certain embodiments, the carrier comprises a diluent, adjuvant, excipient, or vehicle with which probiotic bacteria are administered. In certain embodiments, the carrier can be sterile liquids, such as water and oils, including those of petroleum, animal, vegetable or synthetic origin, such as peanut oil, soybean oil, mineral oil, sesame oil and the like. In certain embodiments, the carrier can be water or aqueous solution, saline solutions and aqueous dextrose and glycerol solutions. In certain embodiments, the carrier can be a solid dosage form carrier, including but not limited to one or more of a binder (for compressed pills), a glidant, an encapsulating agent, a flavorant, and a colorant. Suitable carriers for therapeutic use are well known in the art and are described, for example, in “Remington's Pharmaceutical Sciences” by E. W. Martin, and in “Remington: The Science and Practice of Pharmacy.” Lippincott Williams & Wilkins.

The choice of a carrier can be selected based on the intended route of administration and standard practice. In certain embodiments, oral delivery can be used for delivery to the digestive tract. In certain embodiments, oral formulations comprise additional mixtures, such as milk, yogurt, and infant formula.

In certain embodiments, the duration and frequency of administration can vary between overweight and obese subjects or even between different subjects.

In certain embodiments, solid dosages in the form of tablets are used for the delivery of the probiotic bacteria by mixing the probiotic bacteria with one or more components selected from the group consisting of sodium alginate, calcium carbonate, glyceryl monooleate, triethyl citrate, acetylated monoglyceride, and hypromellose acetate succinate (HPMCAS).

In certain embodiments, the probiotic bacteria or probiotic compositions disclosed herein can be administered parenterally.

In certain embodiments, the probiotic bacteria or probiotic compositions of the presently disclosed subject matter can be prepared for delivery as a solution, a tablet, or as a lyophilized culture. Where cultures are lyophilized, the preparation can be rehydrated in, for example, yogurt or water for administration.

In certain embodiments, the probiotic bacteria or probiotic compositions of the presently disclosed subject matter are formulated such that they can survive passage through the acidic environment of the stomach and such that they adjust quickly to the intestinal environment. Such formulation allows the presently described probiotic bacteria and probiotic compositions to have an elongated half-life in the intestines.

In certain embodiments, the probiotics or probiotic compositions disclosed herein are administered to a subject who has a healthy BMI. In certain embodiments, the probiotics or probiotic compositions disclosed herein are administered to a subject who has a medical condition.

In certain embodiments, the medical condition that can be treated by the presently disclosed probiotics or probiotic compositions is a weight-related condition, including, but not limited to, undernutrition or underweight (Body Mass Index (BMI) of about 18.5 or lower), obesity (BMI of about 30 or higher), overweight (BMI of about 25 to about 30) and associated medical conditions, and/or conditions where imbalanced caloric intake can be a risk factor of certain diseases including, but not limited to, hyperlipidemia, cancer, type 2 diabetes, hypertension, stroke, osteoarthritis, coronary heart disease, sleep apnea and respiratory problems, depression, and gallbladder disease.

In certain embodiments, an obese subject has a BMI of about 30 or higher, an overweight subject has a BMI of about 25 to about 30, a healthy and/or normal subject has a BMI of about 18.5 to about 25, and an underweight subject has a BMI of about 18.5 or lower. In certain embodiments, the probiotics or probiotic compositions disclosed herein are administered to a subject (e.g., a healthy subject) to maintain the body weight of the subject and/or maintaining a BMI of about 18.5 to about 25 of the subject. In certain embodiments, the probiotics or probiotic compositions disclosed herein are administered to a subject to increase the bodyweight of the subject and/or increasing the BMI of the subject who has BMI of about 18.5 or lower. In certain embodiments, the probiotics or probiotic compositions disclosed herein are administered to a subject to reduce the bodyweight of the subject and/or reducing the BMI of the subject who has BMI of about 30 or higher, or about 25 to about 30.

In certain embodiments, the medical condition that can be treated by the presently disclosed probiotics or probiotic compositions is a genetic disorder of carbohydrate metabolism, including, but not limited to, disorders of gluconeogenesis, pyruvate carboxylase deficiency, phosphoenolpyruvate carboxykinase deficiency, and glucose-6-phosphatase deficiency. In certain embodiments, the medical condition that can be treated by the presently disclosed probiotics or probiotic compositions is an eating disorder, including, but not limited to, anorexia nervosa, bulimia nervosa, avoidant/restrictive food intake disorder, and rumination disorder.

In certain embodiments, the medical condition that can be treated by the presently disclosed probiotics or probiotic compositions is hypoglycemia or a pancreatic islet cell tumor. In certain embodiments, the hypoglycemia can be caused by a pancreatic islet cell tumor, e.g., an insulinoma. In certain embodiments, a subject has recurrent hypoglycemic events. In certain embodiments, treating the subject using the presently disclosed probiotics or probiotic compositions reduces the number of hypoglycemic events of the subject.

In certain embodiments, the probiotics or probiotic compositions are administered to the subject in the form of food additives or fortified foods disclosed herein. Dosage of the probiotic bacteria or probiotic composition disclosed herein for the subject (e.g., a subject having a healthy BMI, a subject having an overweight BMI, a subject having an obese BMI, a subject diagnosed with obesity, a subject with an underweight BMI) can vary depending upon the characteristics of the subject (e.g., age, sex, race, weight, height, BMI, body fat percentage, and/or medical history), frequency of administration, manner of administration, clearance of the probiotic bacteria from the subject, and the like.

In certain embodiments, the initial dose can be larger, followed by smaller maintenance doses. In certain embodiments, the dose can be administered as infrequently as weekly or biweekly, or fractionated into smaller doses and administered daily, semi-weekly, etc., to maintain an effective dosage level. In certain embodiments, a variety of doses are effective to achieve colonization of the gastrointestinal tract with the desired probiotic bacterial, for example and not by way of limitation, about 10⁶ CFU, about 10⁷ CFU, about 10⁸ CFU, about 10⁹ CFU, about 10¹⁰ CFU, about 10¹¹ CFU, about 10¹² CFU, about 10¹³ CFU, about 10¹⁴ CFU, or about 10¹⁴ CFU of probiotic bacteria can be administered in a single dose to a subject. In certain embodiments, lower doses can also be effective, for example and not by way of limitation, about 10⁴ and about 10⁵ CFU of probiotic bacteria. In certain embodiments, the probiotic bacteria are administered to a subject in a dosage of between about 10⁶ and about 10⁷ CFU, between about 10⁷ and about 10⁸ CFU, between about 10⁸ and about 10⁹ CFU, between about 10⁹ and about 10¹⁰ CFU, between about 10¹⁰ and about 10¹¹ CFU, between about 10¹¹ and about 10¹² CFU, between about 10¹² and about 10¹³ CFU, between about 10¹³ and about 10¹⁴ CFU, or between about 10¹⁴ and about 10¹⁵ CFU. In certain embodiments, the probiotic bacteria are administered to a subject in a dosage of about 10¹⁰ CFU of probiotics. In certain embodiments, the probiotic bacteria are administered to a subject in a dosage of up to about 10¹² CFU. In certain embodiments, the subject is a human. In certain embodiments, the subject is a domestic animal, e.g., a canine.

In certain embodiments, a probiotic composition or probiotic bacteria disclosed herein can be delivered every 4, 12, 24, 36, 48, 60, or 72 hours. In certain embodiments, the probiotic composition or the probiotic bacteria can be delivered with at least one second pharmaceutically active ingredient, where the second pharmaceutically active ingredient can be delivered simultaneously or sequentially (e.g., within a 4, 12, 24-hour or 1-week period) with the probiotic composition or the probiotic bacteria. In certain embodiments, the probiotic composition or the probiotic bacteria can be delivered with two, three, four, five, or six second pharmaceutically active ingredients. In certain embodiments, the treatment can last for at least about 1 week, at least about 2 weeks, at least about 3 weeks, at least about 4 weeks, at least about 5 weeks, at least about 6 weeks, at least about 2 months, at least about 3 months, at least about 6 months, or at least about 1 year.

In certain embodiments, one or more preparations of different probiotic bacteria can be administered simultaneously (including administering bacteria of the same species or genus, or different species or genus) or sequentially (including administering at different times).

In certain embodiments, the probiotic composition further comprises one or more anti-obesity agent selected from the group consisting of an agent, a therapy, and a pharmaceutically active ingredient that is capable of negatively affecting obesity or weight gain in a subject, for example, by altering one of the fundamental metabolic processes of the host subject's body, as opposed to the probiotic bacteria that themselves have one or more modified fundamental metabolic processes.

In certain embodiments, the second pharmaceutically active ingredient can be an anti-obesity agent. Non-limiting examples of anti-obesity pharmaceutical agents include catecholamine release agents, such as amphetamine, phentermine™ and related substituted amphetamines, agents that increase the human body's metabolism, agents that interfere with the human body's ability to absorb specific nutrients in food, for example and not by way of limitation, ORLISTAT® (tetrahydrolipstatin), loscaserin, sibutramine, rimonabant, METFORMIN™ (N,N-dimethylbiguanide), exenatide, phentermine, as well as herbal and dietary supplements.

In certain embodiments, the probiotic composition further comprises or can be administered in combination with at least one second agent. In certain embodiments, the second agent is an anti-obesity agent. In certain embodiments, the second agent is a weight-gain agent selected from the group consisting of an agent, a therapy, and a pharmaceutically active ingredient that is capable of increasing weight gain in a subject, for example, by introducing high caloric intake. In certain embodiments, the second agent is an anti-depressant agent selected from the group consisting of an agent, a therapy, and a pharmaceutically active ingredient that is capable of relieving symptoms of depression in a subject with eating disorders, for example, by improving the mood and behavior of a subject with anorexia nervosa.

“In combination with,” as used herein, means that the probiotic composition and the at least one second agent are administered to a subject as part of a treatment regimen or plan. In certain embodiments, being used in combination does not require that the probiotic composition and the at least one second agent are physically combined prior to administration or that they be administered over the same time frame. For example, and not by way of limitation, the probiotic composition and the at least one second agent can be administered concurrently to the subject being treated or can be administered at the same time or sequentially in any order or at different points in time.

In certain embodiments, the probiotic compositions disclosed herein comprise probiotics at a concentration of between about 1 weight % and about 100 weight % (% w/w) of the probiotic compositions. In certain embodiments, the probiotic compositions disclosed herein comprise probiotics at a concentration of between about 1 ppm and about 100,000 ppm of the probiotic compositions. In certain embodiments, the probiotic compositions disclosed herein comprise probiotics at a concentration of about 1 pM of the probiotic compositions.

In certain embodiments, the development of personalized probiotic bacteria can allow the use of lower therapeutic amounts due to higher metabolic activity and can further allow the subject to avoid any potential harmful side-effects associated with reintroduction of specific bacterial strains. In certain embodiments, the probiotic bacteria disclosed herein are administered to a different subject or to the same subject. In certain embodiments, the probiotic bacteria administered to the same subject have higher capability of colonizing the intestinal mucosa because of bacteria having already been a part of the gut environment, and immune system recognizes these bacteria as part of the microbiome.

### 5.3 Method of Making

In one aspect, the present disclosure provides methods making probiotics having modified carbohydrate metabolism (e.g., probiotics or probiotic compositions disclosed in Section 5.2). The methods comprises providing bacteria, subjecting the bacteria to a carbohydrate-stress directed evolution to generate the probiotic bacteria, wherein the probiotic bacteria have a modified carbohydrate metabolism as compared to the bacteria, wherein the control probiotic bacteria are not subject to starch-stress directed evolution. In certain embodiments, the bacteria are isolated from a microbiota sample. In one aspect, the present disclosure provides methods making probiotics having modified carbohydrate metabolism comprising providing a microbiota sample, subjecting the microbiota sample to a carbohydrate-stress directed evolution to generate the probiotic bacteria, wherein the probiotic bacteria have a modified carbohydrate metabolism as compared to bacteria in the microbiota sample. In certain embodiments, the probiotic bacteria have a modified carbohydrate metabolism as compared to control probiotic bacteria, wherein the control probiotic bacteria are not subject to starch-stress directed evolution.

Microbiota samples can be obtained and preserved using conventional techniques known in the art. Non-limiting microbiota samples include saliva, tooth swab, tooth scrapping, cheek swabs, throat swab, sputum, endogastric sample, feces, and tissue biopsies. In certain embodiments, the microbiota sample is a saliva sample. In certain embodiments, the microbiota sample is a stool sample. In certain embodiments, bacteria are isolated from the multiple species of microbial flora (e.g. Fungi).

Techniques for the isolation and cultivation of microorganisms (e.g., bacteria) include those, for example, described in the Manual of Clinical Microbiology, 8th edition; American Society of Microbiology, Washington D.C., 2003. Bacterial co-cultures can be cultured according to standard practices. In certain embodiments, techniques for the isolation of the microorganisms can be performed via centrifugation.

In certain embodiments, individual species of the bacteria is isolated using lipolytic agar plates. In certain embodiments, bacteria isolated from the saliva are identified using MALDI-TOF or comparative sequencing of the 16S ribosomal RNA (rRNA) gene in bacteria.

In certain embodiments, the isolated bacteria or the microbiota samples are subject to carbohydrate-stress directed evolution to develop probiotic bacteria having modified carbohydrate metabolism as compared to control bacteria that are not subject to carbohydrate-stress directed evolution. Carbohydrate-stress directed evolution comprises introducing environmental stressors to the bacteria culture to enrich the bacteria for the desired trait, e.g., modified carbohydrate metabolism that favors their survival in the presence of high amount of carbohydrate.

In certain embodiments, the modified carbohydrate metabolism comprises increased glucose production. In certain embodiments, the modified carbohydrate metabolism comprises increased expression or activity of enzymes involved in carbohydrate metabolism.

In certain embodiments, carbohydrate metabolism refers to the breakdown of complex carbohydrates into monosaccharides, disaccharides, and/or oligosaccharides. In certain embodiments, the carbohydrate metabolism refers to metabolizing monosaccharides, e.g., glucose, fructose and galactose. In certain embodiments, the carbohydrate metabolism refers to glycolysis, gluconeogenesis, glycogenolysis, glycogenesis, pentose phosphate pathway, fructose metabolism, and/or galactose metabolism.

In certain embodiments, subject bacteria to carbohydrate-stress directed evolution comprises culturing the bacteria on carbohydrate as the sole energy source. In certain embodiments, subject bacteria to carbohydrate-stress directed evolution comprises culturing the bacteria in increasing levels of carbohydrates.

In certain embodiments, subject bacteria to carbohydrate-stress directed evolution comprises culturing the bacteria on complex carbohydrate as the sole energy source. In certain embodiments, subject bacteria to carbohydrate-stress directed evolution comprises culturing the bacteria in increasing levels of complex carbohydrates.

In certain embodiments, the isolated bacteria are cultured in a culture media having at least about 30%, then in a culture media having at least about 70%, then in a culture media having at least about 100% complexed carbohydrates of total carbohydrates. In certain embodiments, the isolated bacteria are cultured in a culture media having at least about 70%, then in a culture media having at least about 30%, then in a culture media having at least about 100% simple carbohydrates of total carbohydrates.

In certain embodiments, the percentage of complexed carbohydrates of total carbohydrates in the culture is increased by at least about 5%. In certain embodiments, the percentage of complexed carbohydrates of total carbohydrates in the culture is increased by at least about 0.01% increment from 0% to 100%.

In certain embodiments, the carbohydrates are the sole energy sources in the culture media. In certain embodiments, the complex carbohydrate is selected from the group consisting of starches and fibers. In certain embodiments, the complex carbohydrate is a starch or a gelatinized starch. In certain embodiments, the simple carbohydrate is selected from the group consisting of monosaccharides, disaccharides, polyols, glucose, galactose, fructose, xylose, sucrose, lactose, maltose, trehalose, sorbitol, and mannitol. In certain embodiments, the simple carbohydrate is glucose.

In certain embodiments, the probiotic bacteria having modified carbohydrate metabolism can survive in a culture media, wherein at least about 60%, at least about 70%, at least about 80%, at least about 90%, at least about 95%, at least about 99%, or at least about 100% of carbohydrates in the culture media are complex carbohydrates.

In certain embodiments, the probiotic bacteria disclosed herein are optimized for administration to a particular environment, for example, the intestine, a mucosal surface, etc.). That is, in the manufacturing process of a probiotic culture, a combination of microbes is cultured such that they flourish in the gastrointestinal tract of a subject. In certain embodiments, probiotics disclosed herein are also cultured with microbes expected to be in the environment to be treated. Such in vitro conditioning prior to in vivo administration can generate a bacterial culture that is able to survive the milieu of a target site that is contributing to a medical condition.

In certain embodiments, a genetic analysis of the isolated bacteria is performed, to identify lineage and the susceptibility of these bacteria to different antibiotics and to determine pathogenicity.

### 5.4 Methods of Use

The present disclosure relates to methods of regulating body weight and/or glycemia of a subject using probiotics or probiotic compositions having modified carbohydrate metabolism (e.g., probiotics or probiotic compositions disclosed in Section 5.2).

The present disclosure also relates to methods of treating an overweight or obese subject comprising administering the probiotics or probiotic compositions to the subject. The present disclosure also relates to method of treating an underweight subject comprising administering the probiotics or probiotic compositions to the subject. The present disclosure further relates to methods of treating carbohydrate metabolism disorder in a subject comprising administering the probiotics or probiotic compositions to the subject. In certain embodiments, the subject has an underweight BMI, a healthy BMI, an overweight BMI, or an obese BMI. In certain embodiments, the carbohydrate metabolism disorder is selected from the group consisting of diabetes mellitus, lactose intolerance, fructose malabsorption, galactosemia, and glycogen storage disease. The present disclosure also relates to methods of treating an eating disorder in a subject comprising administering the probiotics or probiotic compositions to the subject. In certain embodiments, the eating disorder is anorexia nervosa or bulimia nervosa. The present disclosure also relates to methods of treating hypoglycemia in a subject comprising administering the probiotics or probiotic compositions to the subject. In certain embodiments, the hypoglycemia is caused by cancer.

In certain embodiments, the methods disclosed herein comprise administrating to the subject an effective amount of probiotics disclosed herein. In certain embodiments, the probiotic bacteria disclosed herein reduce carbohydrate absorption by the subject by actively decreasing the amount of carbohydrate available for absorption by the subject. In certain embodiments, the probiotic bacteria disclosed herein increase carbohydrate absorption by the subject by actively increasing the amount of carbohydrate available for absorption by the subject.

In certain embodiments, administering to the subject an effective amount of the probiotic bacteria disclosed herein results in inhibiting weight gain in the subject and/or reducing body weight in the subject. In certain embodiments, administering to the subject an effective amount of the probiotic bacteria disclosed herein results in maintaining a normal BMI of a subject.

In certain embodiments, administering to the subject an effective amount of the probiotic bacteria disclosed herein results in inducing weight gain in the subject. In certain embodiments, administering to the subject an effective amount of the probiotic bacteria disclosed herein results in increasing an underweight BMI of a subject.

In certain embodiments, administering to the subject an effective amount of the probiotic bacteria disclosed herein results in reducing hypoglycemic events in the subject.

In certain embodiments, the probiotic bacteria disclosed herein are administered to the same subject from whom the probiotic bacteria are derived. In certain embodiments, the probiotic bacteria disclosed herein are administered to a different subject from whom the probiotic bacteria are derived.

In certain embodiments, provided herein are methods for treating an overweight or obese subject by administering one or more probiotic compositions disclosed herein comprising one or more probiotic bacteria species, strain, or genus having modified metabolism. In certain embodiments, the treatment can include administration of at least one of probiotic bacteria species or genus included in Table 1 and bacteria species or genus disclosed herein.

In certain embodiments, provided herein are methods for treating various diseases associated with obesity, for example and not by way of limitation, type 2 diabetes, cardiovascular, hypertension, stroke and certain forms of cancer, by administering the probiotic compositions disclosed herein.

In certain embodiments, provided herein are methods for treating an underweight subject by administering one or more probiotic compositions disclosed herein comprising one or more probiotic bacteria species, strain, or genus having modified metabolism.

In certain embodiments, provided herein are methods for treating hypoglycemia subject by administering one or more probiotic compositions disclosed herein comprising one or more probiotic bacteria species, strain, or genus having modified metabolism.

In certain embodiments, the probiotic compositions described herein can be administered in combination with other therapeutic agents or regimes. The choice of therapeutic agents that can be co-administered with the bacterial compositions depends, in part, on the condition being treated. In certain embodiments, the probiotics or probiotic compositions disclosed herein are administered to the subject with at least one anti-obesity agent. In certain embodiments, treatment of an overweight or obese subject can also include one or more conventional regimens including, for example, bariatric surgery.

In certain embodiments, the probiotics or probiotic compositions disclosed herein are administered to the subject with at least one anti-depressant agent.

In certain embodiments, the probiotic bacteria-based treatment regimen can be further supplemented by a dietary change. In certain embodiments, the dietary change includes decreasing dietary fat and sugar consumption, and/or increasing fiber consumption. In certain embodiments, the dietary change includes increasing dietary fat and sugar consumption, and/or increasing vitamins and minerals consumption. In certain embodiments, the probiotic bacteria based treatment regimen can be further supplemented by exercise.

### 5.5 Kits

In non-limiting embodiments, the presently disclosed subject matter provides a kit for administering a probiotic composition of the presently disclosed subject matter (e.g., probiotics or probiotic compositions disclosed in Section 5.2). In certain embodiments, the kit comprises an effective amount of one or more probiotic bacteria species, strain, or genus disclosed herein. In certain embodiments, the kit comprises an effective amount of probiotic bacteria having modified carbohydrate metabolism or a probiotic composition comprising thereof as disclosed herein.

In certain embodiments, the kit can further include one or more components such as instructions for use, devices and additional reagents, and components, such as tubes, containers and syringes for performing the methods disclosed above. In certain embodiments, the kit can further include one or more agents, e.g., anti-obesity agents, that can be administered in combination with the probiotic composition.

In certain embodiments, the kit can include instructions for use, a device for administering the probiotic composition to a subject, or a device for administering an additional agent or compound to a subject. For example, and not by way of limitation, the instructions can include a description of the probiotic composition and, optionally, other components included in the kit, and methods for administration, including methods for determining the proper state of the subject, the proper dosage amount, the proper administration method of administering the probiotic composition, and/or the proper storage of the kit. Instructions can also include guidance for monitoring the subject over the duration of the treatment time.

In certain embodiments, the kit can include a device for administering the probiotic composition and/or one or more agents, e.g., anti-obesity agents to a subject. Any of a variety of devices known in the art for administering medications and probiotic compositions can be included in the kits provided herein. Non-limiting examples of such devices include a hypodermic needle, an intravenous needle, a catheter, a needleless injection device, an inhaler and a liquid dispenser, such as an eyedropper. In certain embodiments, a probiotic composition and/or one or more agents, e.g., anti-obesity agents to a subject to be delivered systemically, for example, by intravenous injection, can be included in a kit with a hypodermic needle and syringe.

In certain embodiments, the kit can comprise one or more containers containing a probiotic composition, disclosed herein. For example, and not by way of limitation, the kit can comprise one or more containers that contain probiotic bacteria comprising at one or more bacteria species, strain, or genus subject to stress-based directed evolution and/or a probiotic composition comprising probiotic bacteria having modified metabolism or a portion thereof.

### 7. EXAMPLES

The presently disclosed subject matter will be better understood by reference to the following Example, which is provided as exemplary of the presently disclosed subject matter, and not by way of limitation.

### Example 1: Generating Bacteria Having Modified Carbohydrate Metabolism

Bacteria having modified carbohydrate metabolism were generated by directed evolution under starch stress from saliva samples collected from human subjects. Saliva samples were collected from four volunteers and mixed. The culture solutions used for starch stress test included glucose source and starch source. Glucose source was BBL broth with 0.2% (w/v) glucose, and starch source was PBS with 0.5% (w/v) gelatinized starch.

In passage 1, 500 mL of saliva samples were cultured in the mixture of 67% (v/v) glucose source+33% (v/v) starch source for 24 h. In passage 2, 100 mL of this culture solution was transferred into the mixture of 33% (v/v) glucose source+67% (v/v) starch source and cultured for 24 h. In passage 3, culture suspension from passage 2 was centrifuged, supernatant was removed, sedimented bacteria was resuspended in starch source (0.5% (w/v) in PBS) and cultured for 24 h. Culture suspension of passage 3 was centrifuged, and supernatant was removed. Sedimented bacteria were resuspended in broth with 30% glycerol and stored at −80° C. as evolved bacteria stocks.

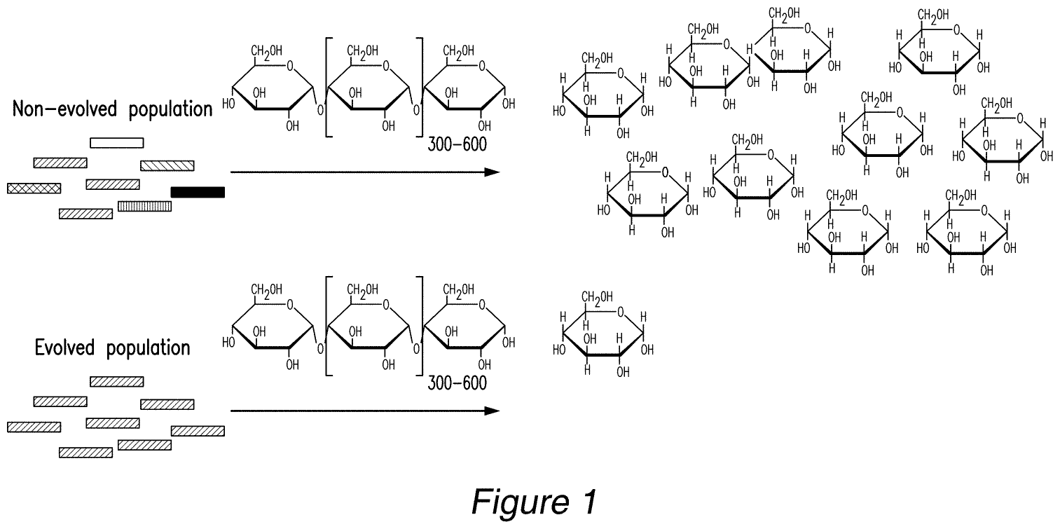

Genomic analysis was performed to identify the bacteria in the evolved bacteria samples. Evolved bacteria samples were spread on starch agar plate prepared in MRS broth. Lugol (I2/KI) solution was poured on the plate to color starch with iodine. Digested starch was not colored and formed a halo. Colonies that generated halo on the plate were picked for 16s rDNA analysis. Streptococcus mitis, Streptococcus pneumoniae, and Streptococcus pseudopneumoniae were found as the most common stains (FIG. 1).

To test the starch consumption in evolved and non-evolved bacteria, frozen stocks from both evolved and non-evolved samples were resuspended in MRS broth with 0.5% (w/v) starch as the only carbohydrate source in the culture medium and cultured for 24 h. Culture suspension was centrifuged to collect supernatant. Bacteria sediment was then resuspended in PBS and plated on starch agar for determining CFUs. The amount of remaining starch in the supernatant was quantified by colorimetric starch assay. It was found that all of the starch in the culture media was consumed by both evolved and non-evolved bacteria for the given culture time. Additionally, the amount of glucose in the supernatant was quantified by colorimetric starch assay, and the results are shown in Table 1.

TABLE 1
Glucose produced by evolved and non-evolved bacteria

| | Glucose | | Glucose |
|---|---|---|---|
| | (mg/ml) | CFU | (mg/mL/CFU) |
| Evolved #1 | 184 | 60 × 10⁷ | 3 × 10⁻⁷ |
| Evolved #2 | 170 | 105 × 10⁷ | 1.6 × 10⁻⁷ |
| Evolved #3 | 277 | 90 × 10⁷ | 3.1 × 10⁻⁷ |
| Un-Evolved #1 | 792 | 4 × 10³ | 0.198 |
| Un-Evolved #2 | 785 | 4 × 10³ | 0.196 |
| Un-Evolved #3 | 773 | 4 × 10³ | 0.06 |

This study shows that evolved bacteria under starch stress consume starch at the same degree with non-evolved bacteria but glucose production is at much smaller degree compared to glucose production from non-evolved ones (FIG. 1).

### Example 2: Generating Bacteria Having Modified Carbohydrate Metabolism

Bacteria having modified carbohydrate metabolism were generated by directed evolution under starch stress from saliva samples collected from C57B^{1/6}j mice. The culture solutions used for the starch stress test included glucose source and starch source. Glucose source was BBL broth with 0.2% (w/v) glucose, and the starch source was PBS with 0.5% (w/v) gelatinized starch.

In passage 1, saliva samples were cultured in the mixture of BBL broth with 0.2% (w/v) glucose to expand for 24 h. In passage 2, 100 μL of this culture solution was transferred into the mixture of 67% (v/v) glucose source+33% (v/v) starch source for 24 h. In passage 3, 100 μL of this culture solution was transferred into the mixture of 33% (v/v) glucose source+67% (v/v) starch source and cultured for 24 h. In passage 4, culture suspension from passage 3 was centrifuged, the supernatant was removed, sedimented bacteria were resuspended in starch source (0.5% (w/v) in PBS) and cultured for 24 h.

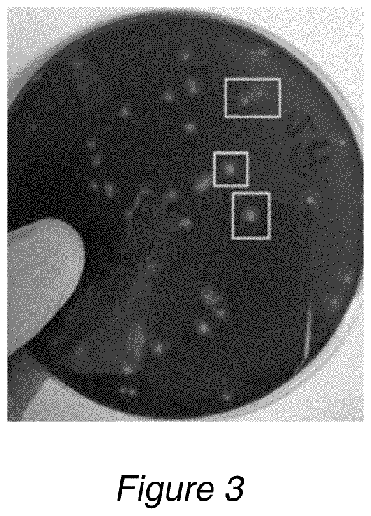

To isolate starch digesting colonies, culture suspension of passage 4 was plated on BHI agar containing starch and glucose. Lugol solution (I₃K) was poured on the plates to color starch with iodine. The bacteria that grew on the plates and digested starch and generated a halo (FIG. 3). Isolated evolved single colonies were expanded in BHI broth with 0.1% (w/v) glucose and 0.1% (w/v) starch.

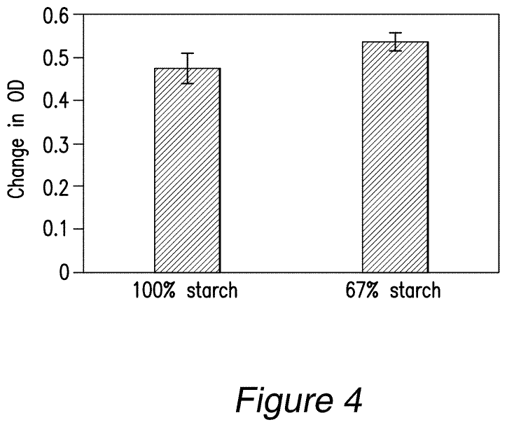

To test the survival of the evolved bacteria during and after the evolution process, samples from passage 3 (67% starch) and evolved frozen samples (100% starch) were incubated in BHI broth for 7 h. Optical densities (OD) of the cultures were measured before and after the incubation. Changes in optical densities illustrated the survival of bacteria during (67% starch) and after (100% starch) the evolution process (FIG. 4).

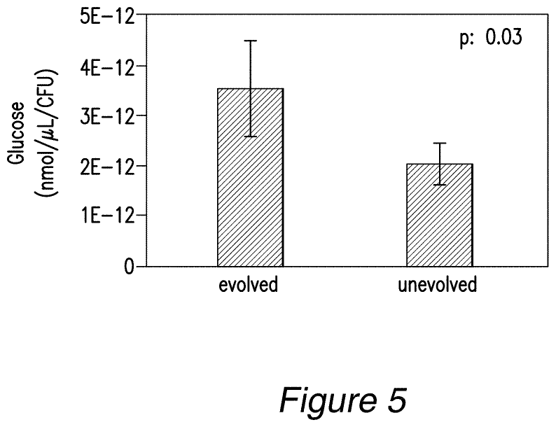

To test the carbohydrate metabolism of the evolved bacteria, glucose production was determined. Frozen stock from both evolved and non-evolved samples were resuspended in BHI broth with 0.1% (w/v) starch and 0.1% (w/v) glucose for expanding for 24 h. The culture suspension was centrifuged, and the supernatant was collected. The bacteria sediment was resuspended in PBS with 0.05% starch and optical densities were measured for estimation of bacteria-count expressed as colony forming units (CFU). The samples were cultured for 7 h in this solution. The bacteria were removed by centrifugation and the culture supernatant was used to quantify glucose production by colorimetric glucose assay. The evolved bacteria showed modified carbohydrate metabolism and a change in glucose production when compared to the unevolved bacteria (FIG. 5). Without being bound to any particular theory, these data show that the probiotics of the disclosed subject matter were able to change glucose production and had modified carbohydrate metabolism.

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes

What is claimed is:

1. A probiotic composition comprising probiotic bacteria, wherein the probiotic bacteria have a modified carbohydrate metabolism.

2. The probiotic composition of claim 1, wherein the probiotic bacteria produce a change in glucose production as compared to control probiotic bacteria.

3. The probiotic of claim 1, wherein the probiotic bacteria increase carbohydrate absorption by a host subject.

4. The probiotic composition of claim 1, wherein the probiotic bacteria reduce carbohydrate absorption by a host subject.

5. The probiotic composition of claim 1, wherein the probiotic bacteria survive in a culture media, wherein at least about 60% of carbohydrates in the culture media are starches.

6. The probiotic composition of claim 5, wherein at most about 40% of carbohydrates in the culture media are sugars.

7. A kit comprising the probiotic composition of claim 1.

8. A method of making a probiotic composition comprising:
(a) subjecting a microbiota sample of a subject to a starch-stress directed evolution to generate probiotic bacteria, wherein the probiotic bacteria have a modified carbohydrate metabolism as compared to control probiotic bacteria, wherein the control probiotic bacteria are not subject to starch-stress directed evolution; and
(b) incorporating the probiotic bacteria to the probiotic composition.

9. The method of claim 8, wherein the microbiota sample is a saliva sample or a stool sample.

10. The method of claim 7, wherein the probiotic bacteria increase carbohydrate absorption by a subject.

11. The method of claim 7, wherein the probiotic bacteria reduce carbohydrate absorption by a subject.

12. The method of claim 7, wherein the probiotic bacteria survive in a culture media, wherein at least about 60% of carbohydrates in the culture media are starches.

13. The method of claim 11, wherein at most about 40% of carbohydrates in the culture media are sugars.

14. The method of claim 7, wherein the starch-stress directed evolution comprises culturing the microbiota sample in increasing starch concentrations.

15. A method of treating a metabolic disorder in a subject in need thereof, comprising:
(a) subjecting a microbiota sample of the subject to a starch-stress directed evolution to generate probiotic bacteria, wherein the probiotic bacteria have a modified carbohydrate metabolism as compared to control probiotic bacteria, wherein the control probiotic bacteria are not subject to starch-stress directed evolution; and
(b) administering to the subject an effective amount of the probiotic bacteria.

16. The method of claim 15, wherein the probiotic bacteria increase carbohydrate absorption by the subject.

17. The method of claim 15, wherein the probiotic bacteria reduce carbohydrate absorption by the subject.

18. The method of claim 15, wherein the probiotic bacteria survive in a culture media, wherein at least about 60% of carbohydrates in the culture media are starches.

19. The method of claim 18, wherein at most about 40% of carbohydrates in the culture media are sugars.

20. The method of claim 15, wherein the starch-stress directed evolution comprises culturing the microbiota sample in increasing starch concentrations.
